(19) United States
(12) Patent Application Publication
(10) Pub. No.: US 2024/0139077 A1
(43) Pub. Date: May 2, 2024

(54) TREATING DENTAL CARIES

(71) Applicant: Solcarry Ltd., Netanya (IL)

(72) Inventors: Hila Lin, Tel Aviv (IL); Yael Helfman Cohen, Zichron Yaakov (IL); Andreana Piancastelli, Faenza (IL)

(73) Assignee: Solcarry Ltd., Netanya (IL)

(21) Appl. No.: 18/549,393

(22) PCT Filed: Mar. 1, 2022
(86) PCT No.: PCT/IB2022/051769

§ 371 Date: Sep. 7, 2023

Related U.S. Application Data
(60) Provisional application No. 63/160,011, filed on Mar. 12, 2021.

(51) Int. Cl. A61K 6/884; A61K 6/17; A61K 6/70

(52) U.S. Cl. CPC A61K 6/884; A61K 6/17; A61K 6/70

(57) ABSTRACT

A method includes introducing an encapsulation material through an opening, such as a decay hole or porosity, in a tooth, the encapsulating material reaching and at least partially encapsulating caries in the tooth.

### FIELD OF THE INVENTION

The present invention relates generally to treatment of caries, particularly to atraumatic treatment of caries by encapsulation of the caries.

### BACKGROUND OF THE INVENTION

Dental caries is a sugar-driven, dynamic disease that results in the demineralization of dental hard tissues, generally due to acids made by oral bacteria. Treatment of caries typically requires drilling into the tooth to reach and remove the caries. The drilled portion is typically filled with a filling. Fillings (restorations) are the main treatment option when decay has progressed beyond the earliest stage. Fillings are made of various materials, such as tooth-colored composite resins, porcelain or dental amalgam that is a combination of several materials. If the decay is more advanced, crowns may be necessary.

The drilling process can damage healthy tissues and cause anxiety to patients. After drilling to the cavity, the tooth is mechanically weakened.

### SUMMARY OF THE INVENTION

The present invention seeks to provide novel materials and methods for atraumatic treatment of caries by encapsulation of the caries, as is described more in detail hereinbelow. There is no drilling and removal of the caries; instead the caries is neutralized by encapsulation. “Caries” includes any kind of decay, such as caries, secondary caries, recurrent caries and so forth.

There is provided in accordance with an embodiment of the invention a method including introducing an encapsulation material through a decay hole in a tooth, the encapsulating material reaching and at least partially encapsulating caries in the tooth. The encapsulation material may include antibacterial nanoparticles for destroying the caries.

The encapsulation material may create an encapsulated intermediate layer, and the intermediate layer may be used as a scaffold for tooth rehabilitation.

In accordance with an embodiment of the invention the tooth rehabilitation includes causing a restoration material to adhere to the intermediate layer of the encapsulation material, wherein the intermediate layer adheres to the caries and to the restoration material. The intermediate layer adheres to at least one of dentin and enamel in the tooth.

Prior to introducing the encapsulation material, a porosity increasing material may be applied to or near the caries to increase porosity of a surface area of the caries.

In accordance with an embodiment of the invention the encapsulation material may penetrate into the caries through porous surfaces at or near the caries. The encapsulation material may harden, solidify or dry the caries. The encapsulation material may cause remineralization of the caries. The encapsulation material may cause crystallization of an outer surface of the caries.

The time it takes to apply the encapsulation material is short, in the range of a few seconds to minutes. The encapsulation material remains effective for a long time, that is, it remains active and anti-bacterial, and retains its bonding properties for a long time, typically for many years and perhaps for the entire tooth lifetime, depending on clinical trial results.

Restoration materials may undergo contraction after application to the tooth. This contraction may cause small gaps which can cause “leakage” or “leaky fillings”, that is, new invasion of germs into the tooth, which can cause secondary caries. The encapsulation and restoration materials of the invention do not increase leakage and comply with dental standards for leakage.

The bonding characteristics of the encapsulation material match or exceed bonding characteristics of known prior art dental bonding materials.

The encapsulation and restoration materials of the invention withstand biting forces of the patient. The encapsulation and restoration materials of the invention do not cause any sensitivity in the tooth or mouth, and even reduce any sensitivity or pain compared to the prior art which, unlike the present invention, uses drilling.

The encapsulation and restoration materials of the invention provide an effective solution to a wide variety of caries, including but not limited to, primary caries, advanced caries, proximal caries, deep caries and secondary caries. In certain cases of proximal caries between adjacent teeth, it is contemplated that minor drilling may be needed to in order to introduce the materials.

There is provided in accordance with an embodiment of the invention a dental material including a medically safe encapsulation material that has capability of flowing through an opening, such as a decay hole or porosity, in a tooth to caries in the tooth and at least partially encapsulating the caries, the encapsulation material being capable of adhering to at least one of dentin and enamel in the tooth.

In another aspect of the invention, the encapsulation and/or restoration materials of the invention may be added to a mouth wash or dentifrice as a preventive to tooth decay.

### BRIEF DESCRIPTION OF THE DRAWINGS

The present invention will be understood and appreciated more fully from the following detailed description taken in conjunction with the drawings in which:

FIGS. 1, 2, 3 are simplified pictorial illustrations of encapsulation material being introduced into a decay opening in enamel, dentin and pulp, respectively, of a tooth, so that the encapsulation material encapsulates caries, in accordance with a non-limiting embodiment of the present invention;

FIGS. 4A and 4B are simplified pictorial illustrations, respectively, before and after encapsulation of the caries;

FIGS. 5A and 5B are simplified pictorial illustrations, respectively, before and after encapsulation of the caries;

FIGS. 6A and 6B are different magnified illustrations of SEM (scanning electron microscope) cross-section of the tooth with the encapsulation material;

FIGS. 7A and 7B are different magnified illustrations of SEM micrographs of the interface of a tooth, wherein the encapsulation material has bonded both to the caries and to the composite restoration material; and

FIGS. 8A and 8B are comparisons of SEM micrographs of the tooth having caries, without treatment with the encapsulation material (FIG. 8A) and with treatment with the encapsulation material (FIG. 8B).

### DETAILED DESCRIPTION

Reference is now made to FIGS. 1-3, which illustrate a method for non-drilling treatment of dental caries, in accordance with a non-limiting embodiment of the present invention.

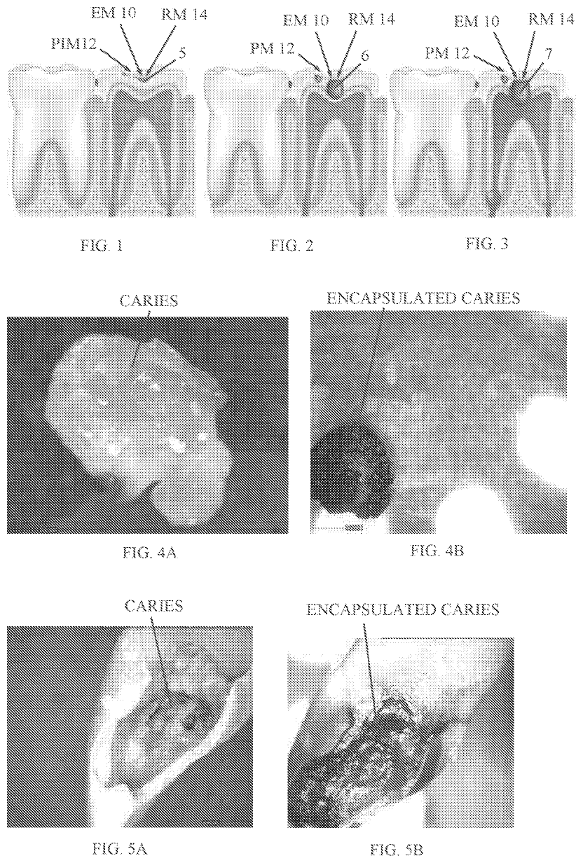

In FIG. 1, decay has formed on the enamel of the tooth. The decay forms an opening 5. In FIG. 2, decay has progressed to the dentin of the tooth and forms an opening 6. In FIG. 3, decay has progressed to the pulp of the tooth and forms an opening 7. In an embodiment of the invention, an encapsulation material 10 is introduced into opening 5, 6 or 7. The invention exploits the existing decay opening 5, 6 or 7; there is no need to drill a new hole or even just a small hole (interproximal hole).

The encapsulation material 10 (designated EM in the figures) may be injected or otherwise introduced into opening 5, 6 or 7 in one or more stages. For example, the encapsulation material 10 may be introduced by a dropper, dripper, injection (any kind, including micro-injection), syringe, wash, spray, and other methods. Prior to introduction of material 10, the caries surface may be prepared by dripping or otherwise applying a porosity increasing material 12 (designated PIM in the figures), such as but not limited to, polyethylene imine (PEI) which increases the porosity of the caries surface area. The encapsulation material 10 encapsulates the caries and destroys the caries by blocking their access to sugars. Additionally or alternatively, the encapsulation material 10 includes antibacterial nanoparticles for destroying the caries.

Examples of encapsulation material 10 include, without limitation, a polymer, protein or hybrid material, which has the ability to adhere to both wet and dry surfaces upon contact, for example, through intermolecular forces such as hydrogen bonds, electrostatic interactions or van der Waals interactions or any combination thereof. This characteristic can be very challenging when dealing with wet surfaces (as with caries) because water separates the molecules of the two surfaces, preventing interactions. Adhesives can suffer from several limitations, such as weak bonding, low biological compatibility, poor mechanical strength, and slow adhesion formation. The encapsulation material used in the invention acts as an adhesive to a wet surface (caries) and a dry surface (restoration composite, enamel, or dentin), and matches or exceeds the bonding characteristics of known dental bonding materials, and the adhesion formation is relatively fast.

The encapsulation material can be modified to bond to external materials, such as but not limited to, through intermolecular forces, to specific sites that can give it different properties, such as antibacterial activity, regeneration of enamel or improved mechanical strength, and other properties.

One non-limiting example of encapsulation material is an oligomeric protein. Oligomeric proteins, by definition, are composed of more than one subunit (polypeptide chain). Oligomeric proteins may be composed either exclusively of several copies of identical polypeptide chains, in which case they are termed porno-oligomers, or alternatively by at least one copy of different polypeptide chains (hetero-oligomers).

The encapsulation material can be bonded with antibacterial nanoparticles, such as but not limited to, zinc oxide, titanium oxide or yttrium fluoride, or any combination thereof. The encapsulation material 10 may include tooth building blocks, such as but not limited to, calcium ions, fluoride ions and hydroxyapatite, or any combination thereof.

The encapsulation material 10 may be a clear liquid that is dripped over the decay area of the tooth in a single or multi-stage treatment and forms an intermediate layer. The shelf-life of encapsulation material 10 may be, without limitation, a year or more.

The caries is now encapsulated and blocked in an encapsulated intermediate layer. This intermediate layer can now be used as a scaffold for tooth rehabilitation. Any suitable restoration material or materials 14 (designated RM in the figures) may be injected or otherwise introduced into the opening and adhere to the intermediate layer of the encapsulation material 10.

The intermediate layer of the encapsulation material 10 adheres both to the caries and to the restoration material 14 (or bonding material). In contrast, prior art off-the-shelf dental materials do not adhere to caries in dentin because dentin is soft and wet. The encapsulation material 10 has strong adherence to dentin, enamel and cementum.

Accordingly, the encapsulation material 10 fully or partially encapsulates the caries, creates an intermediate barrier layer, and adheres to the restoration material 14. The encapsulation material 10 may penetrate into the caries through porous surfaces. The encapsulation material 10 may harden, solidify or dry up the caries, or cause remineralization of the caries, to a level of healthy teeth. The process (particularly drying of the caries and/or crystallization of the outer surface of the caries) may enhance the adhesion between the caries and the restorative material 14 by the additional mechanical structure through the micro-pores in the caries.

The whole process does not require drilling or anesthesia. The process can eliminate the need of several visits to the dentist by treating the entire mouth at once.

FIGS. 4A/4B and 5A/5B illustrate the use of the encapsulating material, respectively, before and after encapsulation of the caries.

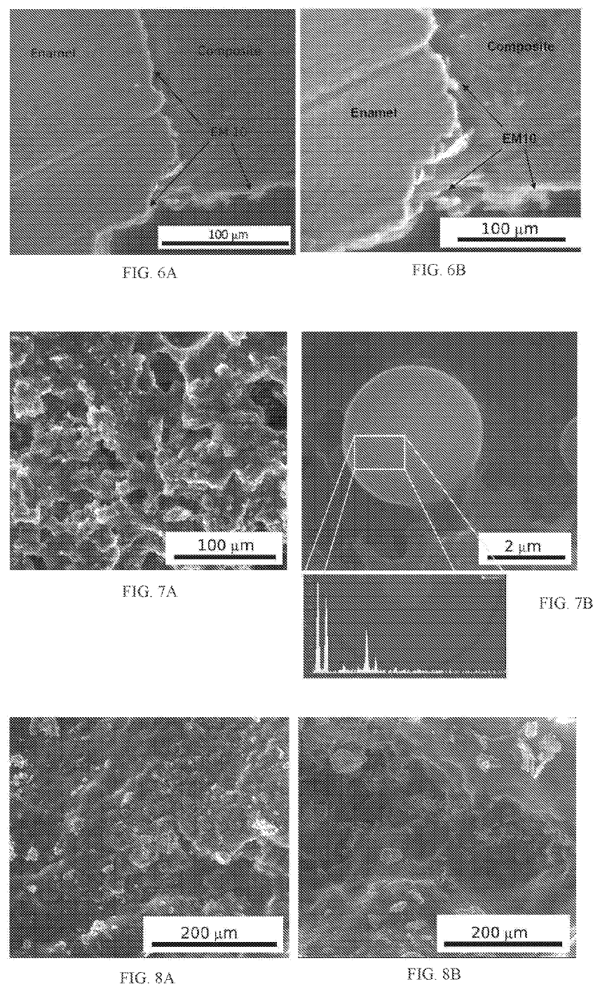

FIGS. 6A and 6B are different magnified illustrations of SEM (scanning electron microscope) cross-section of the tooth with the encapsulation material (EM) 10. It can be seen that the EM 10 coats the interface between the enamel and the composite material (the bright layer). The composite layer is characterized with Si (silicon) spheres all over the surface.

FIGS. 7A and 7B are different magnified illustrations of SEM micrographs of the interface of a tooth, wherein EM 10 has bonded both to the caries and to the composite restoration material. FIG. 7B also shows graphically the strong presence of Si spectral lines.

FIGS. 8A and 8B are comparisons of SEM micrographs of the tooth having caries, without treatment with EM 10 (FIG. 8A) and with treatment with EM 10 (FIG. 8B). It can be seen that the caries without the treatment looks very messy with crystalline structure, whereas the caries with the treatment is coated and more organized.

What is claimed is:

1. A method comprising:
introducing an encapsulation material through an opening in a tooth, said encapsulating material reaching and at least partially encapsulating caries in the tooth.

2. The method according to claim 1, wherein said opening is a decay hole or a porosity.

3. The method according to claim 1, wherein said encapsulation material comprises antibacterial nanoparticles for destroying the caries.

4. The method according to claim 1, wherein said encapsulation material creates an encapsulated intermediate layer, and the method further comprises using said intermediate layer as a scaffold for tooth rehabilitation.

5. The method according to claim 1, wherein said tooth rehabilitation comprises causing a restoration material to adhere to said intermediate layer of said encapsulation material, wherein said intermediate layer adheres to the caries and to said restoration material.

6. The method according to claim 5, wherein said intermediate layer adheres to at least one of dentin and enamel in the tooth.

7. The method according to claim 1, wherein prior to introducing said encapsulation material, a porosity increasing material is applied to or near said caries to increase porosity of a surface area of the caries.

8. The method according to claim 1, wherein said encapsulation material penetrates into the caries through porous surfaces at or near the caries.

9. The method according to claim 1, wherein said encapsulation material hardens, solidifies or dries the caries.

10. The method according to claim 1, wherein said encapsulation material causes remineralization of the caries.

11. The method according to claim 1, wherein said encapsulation material causes crystallization of an outer surface of the caries.

12. A dental material comprising:
a medically safe encapsulation material that has capability of flowing through an opening in a tooth to caries in the tooth and at least partially encapsulating the caries, said encapsulation material being capable of adhering to at least one of dentin and enamel in the tooth.

13. The dental material according to claim 12, wherein said encapsulation material comprises antibacterial nanoparticles for destroying the caries.

14. The dental material according to claim 12, wherein said encapsulation material is capable of penetrating into the caries through porous surfaces at or near the caries.

15. The dental material according to claim 12, wherein said encapsulation material is capable of hardening, solidifying or drying the caries.

16. The dental material according to claim 12, wherein said encapsulation material is capable of causing remineralization of the caries.

17. The dental material according to claim 12, wherein said encapsulation material is capable of causing crystallization of an outer surface of the caries.
